(19) United States
(12) Patent Application Publication
(10) Pub. No.: US 2024/0374880 A1
(43) Pub. Date: Nov. 14, 2024

(54) CONVENIENT-TO-ASSEMBLE ESOPHAGUS MEDICINE APPLYING DEVICE FOR THE GASTROENTEROLOGY DEPARTMENT

(71) Applicant: GUANGDONG GUOSHIJIAN TECHNOLOGY DEVELOPMENT CO., LTD., Foshan, Guangdong (CN)

(72) Inventors: Wei MA, Foshan, Guangdong (CN); Lihua SHENG, Foshan, Guangdong (CN)

(21) Appl. No.: 18/033,413

(22) PCT Filed: Dec. 30, 2021
(86) PCT No.: PCT/CN2021/143148

§ 371 Date: Apr. 24, 2023

(30) Foreign Application Priority Data
Nov. 17, 2021 (CN) 202111364806.5
Dec. 30, 2021 (WO) PCT/CN2021/143148

(51) Int. Cl. A61M 35/00

(52) U.S. Cl. CPC A61M 35/00; A61M 2202/0007; A61M 2202/04; A61M 2205/0216; A61M 2205/10; A61M 2205/8206

(57) ABSTRACT

A esophagus medicine applying device of the present invention includes a shell, an applying shell, an assembling mechanism, a return mechanism, a power supply mechanism, an arc cover, a camera, a transparent cover and an end cover; the return mechanism is arranged within the shell, the assembling mechanism is arranged on the applying shell, and the power supply mechanism is arranged on the shell; the arc cover is connected to the applying shell through threads, the camera is fixedly connected to the interior of the arc cover, and the transparent cover is fixedly connected to the arc cover; one end of the shell is in threaded connection with the end cover, a partition plate is fixedly connected to the interior of the shell, an input pipe is fixedly connected to the interior of the shell, and an output pump is fixedly connected to the partition plate.

### TECHNICAL FIELD

This invention generally relates to the technical field of gastroenterology, and more particularly, to a convenient-to-assemble esophagus medicine applying device for the gastroenterology department.

### BACKGROUND

Gastroenterology is a third-level clinical discipline for studying diseases of esophagus, stomach, small intestine, large intestine, liver, gallbladder and pancreas. Gastroenterology relates to various diseases, wide range of medical knowledge and complex operations. The esophagus is a muscular passageway that connects the throat to the stomach, which helps transporting food into the stomach but does not possess any digestive function. Esophagitis is a common disease in gastroenterology department. When doctors diagnose and treat esophagitis, medicines are normally applied to the esophagus to ensure a patient's rapid recovery,. However, conventional medicine applying devices have the following shortcomings: 1. the device needs to be disassembled and cleaned which can be quite inconvenient: 2. excessive liquid medicine cannot be recycled during the medical application, leading to the waste of liquid medicine: 3. when the device needs to be used at various positions, the battery is inconvenient to replace, resulting in the inconvenient use of the device. Therefore, it is urgent for those skilled in the art to develop a convenient-to-assemble esophagus medicine applying device for the gastroenterology department.

### SUMMARY

The purpose of the present invention is to provide a convenient-to-assemble esophagus medicine applying device for the gastroenterology department.

To achieve the above purpose, the present invention adopts the following technical solution:

A convenient-to-assemble esophagus medicine applying device for the gastroenterology department of the present invention comprising a shell, an applying shell, an assembling mechanism, a return mechanism, a power supply mechanism, an arc cover, a camera, a transparent cover and an end cover: the return mechanism is arranged within the shell, the assembling mechanism is arranged on the applying shell, and the power supply mechanism is arranged on the shell; the arc cover is connected to the applying shell through threads, the camera is fixedly connected to the interior of the arc cover, and the transparent cover is fixedly connected to the arc cover; one end of the shell is in threaded connection with the end cover, a partition plate is fixedly connected to the interior of the shell, an input pipe is fixedly connected to the interior of the shell, and an output pump is fixedly connected to the partition plate.

In another preferred embodiment of the present invention, the assembling mechanism comprises an infusion pipe, a through hole, an applying sponge, a driving ring, a first spring, a connecting column, a corrugated pipe, an inclined surface block and a first sealing ring. The infusion pipe is fixedly connected to the interior of the applying shell, the through hole is formed in the infusion pipe, and the applying sponge is fixedly connected to the outer side of the infusion pipe. The applying sponge is in contact with the applying shell, the driving ring is slidably connected to the infusion pipe, the first spring is arranged on the driving ring, the connecting column is slidably connected to the interior of the infusion pipe, the inclined surface block is fixedly connected to the connecting column, and the inclined surface block is slidably connected to the driving ring. The corrugated pipe is arranged on the outer side of the connecting column, the first sealing ring is attached to the interior of the infusion pipe, and the first sealing ring is in contact with the input pipe. The input pipe is slidably connected to the infusion pipe, and the infusion pipe is in contact with the inclined surface block.

In another preferred embodiment of the present invention, one end of the first spring is fixedly connected to the driving ring, and the other end of the first spring is fixedly connected to the infusion pipe.

In another preferred embodiment of the present invention, one end of the corrugated pipe is fixedly connected to the inclined surface block, and the other end of the corrugated pipe is fixedly connected to the infusion pipe.

In another preferred embodiment of the present invention, the return mechanism comprises a return ring, a return pipe, a threaded cover, a second sealing ring, a sealing plate, a Y-shaped pipe, a threaded pipe, a protective cover, a return pump and a filter screen. The return ring is fixedly connected to the shell, the return pipe is slidably connected to the return ring, and the threaded cover is movably connected to an outer side of the return pipe. The threaded cover is in threaded connection with the return ring, the Y-shaped pipe is fixedly connected to the partition plate, and the threaded pipe is connected to the outer side of the Y-shaped pipe through threads. The return pump is fixedly connected to the threaded pipe, and the return pump is fixedly connected to the Y-shaped pipe. The protective cover is fixedly connected to the outer side of the threaded pipe, the filter screen is fixedly connected to the protective cover, and the Y-shaped pipe is fixedly connected to the shell and the return ring.

In another preferred embodiment of the present invention, a second sealing ring is attached to the return ring, and the second sealing ring is in contact with the return pipe.

In another preferred embodiment of the present invention, the return pipe is fixedly connected to the applying shell, and the sealing plate is fixedly connected to the interior of the applying shell. The sealing plate and the return pipe are both in contact with the applying sponge, and the return pipe is fixedly connected to the sealing plate.

In another preferred embodiment of the present invention, the power supply mechanism comprises a power supply handle compartment, a conductive plate, a second spring, a conductive cover, a conductive elastic piece, a screw, a baffle and a dry battery compartment. The power supply handle compartment is fixedly connected to an outer side of the shell, the conductive plate is slidably connected to the interior of the power supply handle compartment, and the second spring is arranged on the conductive plate. The conductive cover is hinged to the power supply handle compartment, the conductive elastic piece is fixedly connected to the conductive cover, and the screw is slidably connected to the interior of the conductive cover. The baffle is fixedly connected to an outer side of the screw, the screw is in threaded connection with the power supply handle compartment, and the baffle is in contact with the power supply handle compartment. The dry battery compartment is slidably connected to the interior of the power supply handle compartment, and the dry battery compartment is in contact with the conductive plate and the conductive elastic piece. One end of the second spring is fixedly connected with the power supply handle compartment, and the other end of the second spring is fixedly connected to the conductive plate.

Compared with the prior art, the present invention has the following advantages:

The present invention provides a convenient-to-assemble esophagus medicine applying device for the gastroenterology department, which achieves advantages including convenient assembly, easy recycle of excessive liquid medicine and convenient replacement of battery: when in use, compared with traditional esophagus medicine applying devices for the gastroenterology department, the device of the present invention achieves the following benefits:

First, by means of the return mechanism arranged on the shell, the excessive medicine in the applying sponge is absorbed through the return pipe in the return mechanism, and the raw material is discharged into the shell through the Y-shaped pipe, so that the waste of the medicine is effectively avoided:

Second, through the driving ring, the first spring, the inclined surface block and other structures arranged on the input pipe, when the device needs to be disassembled, the driving ring is separated from the inclined surface block, which allows the connecting column to be separated from input pipe through the corrugated pipe, so that a convenient disassembly is achieved: when the device needs to be assembled, the input pipe is inserted into the interior of the infusion pipe, and the driving ring is released, so that the input pipe is fixed by the connecting column: moreover, when the device is lack of power, the dry battery compartment is conveniently replaced.

### BRIEF DESCRIPTION OF THE DRAWINGS

For ease of illustration, the present invention is described in detail by the following embodiments and drawings.

FIG. 1 is a schematic diagram illustrating an overall three-dimensional structure of the present invention;

FIG. 2 is a schematic diagram illustrating a top view of the shell in FIG. 1 of the present invention;

FIG. 3 is a schematic diagram illustrating a front sectional view of the power supply handle compartment in FIG. 1 of the present invention;

FIG. 4 is a schematic diagram illustrating an enlarged structure of portion A in FIG. 2 of the present invention;

FIG. 5 is a schematic diagram illustrating an enlarged structure of portion B in FIG. 2 of the present invention;

FIG. 6 is a schematic diagram illustrating an enlarged structure of portion C in FIG. 2 of the present invention;

FIG. 7 is a schematic diagram illustrating an enlarged structure of portion D in FIG. 3 of the present invention;

In Figures: 1-Shell, 2-Applying Shell, 3-Assembling Mechanism, 4-Return Mechanism, 5-Power Supply Mechanism, 6-Arc Cover, 7-Camera, 8-Transparent Cover, 9-End Cover, 10-Partition Plate, 12-Output Pump, 13-Input Pipe, 31-Infusion Pipe, 32-Through Hole, 33-Applying Sponge, 34-Driving Ring, 35-The First Spring, 36-Connecting Column, 37-Corrugated Pipe, 38-Inclined Surface Block, 39-The First Sealing Ring, 41-Return Ring, 42-Return Pipe, 43-Threaded Cover, 44-The Second Sealing Ring, 45-Sealing Plate, 46-Y-shaped Pipe, 47-Threaded Pipe, 48-Protective Cover, 49-Return pump, 40-Filter Screen, 51-Power Supply Handle Compartment, 52-Conductive Plate, 53-The Second Spring, 54-Conductive Cover, 55-Conductive Elastic Piece, 56-Screw, 57-Baffle, 58-Dry Battery Compartment.

### DETAILED DESCRIPTION

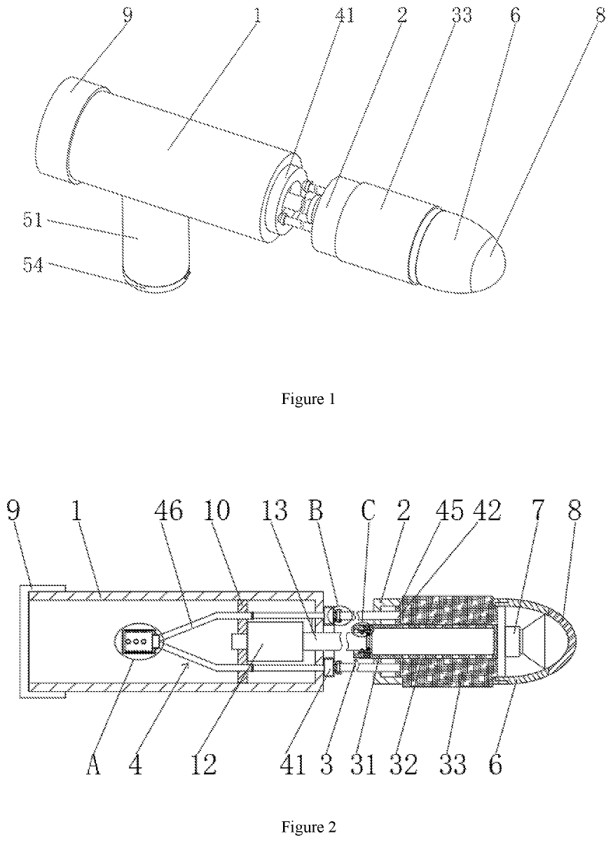

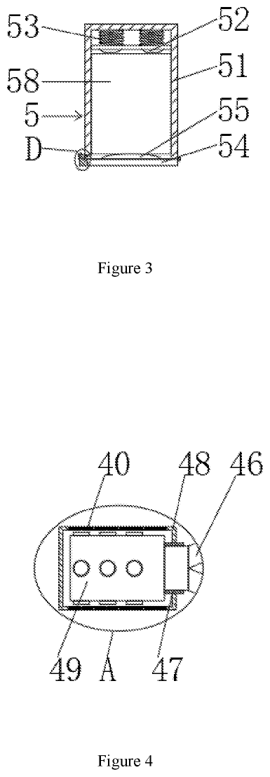

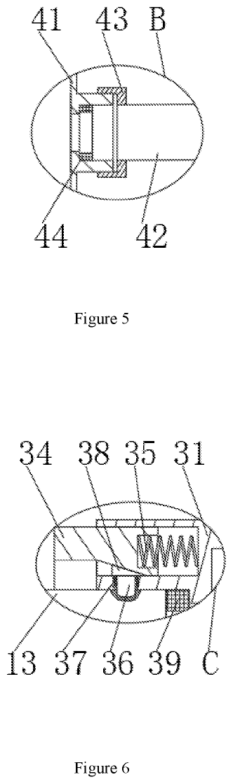

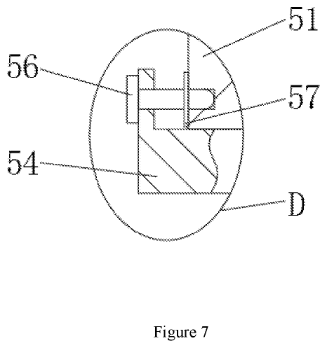

As shown in FIGS. 1-7, the embodiment of the present invention adopts the following technical solution:

### Embodiment 1

The convenient-to-assemble esophagus medicine applying device for the gastroenterology department of the present invention comprises a shell 1, an applying shell 2, an assembling mechanism 3, a return mechanism 4, a power supply mechanism 5, an arc cover 6, a camera 7, a transparent cover 8 and an end cover 9. The return mechanism 4 is arranged within the shell 1, the assembling mechanism 3 is arranged on the applying shell 2, and the power supply mechanism 5 is arranged on the shell 1. The arc cover 6 is connected to the applying shell 2 through threads, the camera 7 is fixedly connected to the interior of the arc cover 6, and the transparent cover 8 is fixedly connected to the arc cover 6. One end of the shell 1 is in threaded connection with the end cover 9, a partition plate 10 is fixedly connected to the interior of the shell 1, an input pipe 13 is fixedly connected to the interior of the shell 1, and an output pump 12 is fixedly connected to the partition plate 10.

Further, the assembling mechanism 3 comprises an infusion pipe 31, a through hole 32, an applying sponge 33, a driving ring 34, a first spring 35, a connecting column 36, a corrugated pipe 37, an inclined surface block 38 and a first sealing ring 39. The infusion pipe 31 is fixedly connected to the interior of the applying shell 2, the through hole 32 is formed in the infusion pipe 31, and the applying sponge 33 is fixedly connected to the outer side of the infusion pipe 31. The applying sponge 33 is in contact with the applying shell 2, the driving ring 34 is slidably connected to the infusion pipe 31, the first spring 35 is arranged on the driving ring 34, the connecting column 36 is slidably connected to the interior of the infusion pipe 31, the inclined surface block 38 is fixedly connected to the connecting column 36, and the inclined surface block 38 is slidably connected to the driving ring 34. The corrugated pipe 37 is arranged on the outer side of the connecting column 36, the first sealing ring 39 is attached to the interior of the infusion pipe 31, and the first sealing ring 39 is in contact with the input pipe 13. The first sealing ring 39 and the input pipe 13 are in a compressed state, and the input pipe 13 is slidably connected to the infusion pipe 31. An inclined surface is provided on the inner side of the driving ring 34, and the infusion pipe 31 is in contact with the inclined surface block 38.

Furthermore, one end of the first spring 35 is fixedly connected to the driving ring 34, the other end of the first spring 35 is fixedly connected to the infusion pipe 31, and the first spring 35 is capable of propelling the driving ring 34 to automatically reset through the elastic force.

One end of the corrugated pipe 37 is fixedly connected to the inclined surface block 38, the other end of the corrugated pipe 37 is fixedly connected to the infusion pipe 31, and the corrugated pipe 37 is capable of propelling the infusion pipe 31 to automatically disengage from the input pipe 13 by means of the elasticity.

In addition, the return mechanism 4 comprises a return ring 41, a return pipe 42, a threaded cover 43, a second sealing ring 44, a sealing plate 45, a Y-shaped pipe 46, a threaded pipe 47, a protective cover 48, a return pump 49 and a filter screen 40. The return ring 41 is fixedly connected to the shell 1, the return pipe 42 is slidably connected to the return ring 41, and the threaded cover 43 is movably connected to an outer side of the return pipe 42. The threaded cover 43 is in threaded connection with the return ring 41, the Y-shaped pipe 46 is fixedly connected to the partition plate 10, and the threaded pipe 47 is connected to the outer side of the Y-shaped pipe 46 through threads. The return pump 49 is fixedly connected to the threaded pipe 47, and the return pump 49 is fixedly connected to the Y-shaped pipe 46. The protective cover 48 is fixedly connected to the outer side of the threaded pipe 47, the filter screen 40 is fixedly connected to the protective cover 48, the Y-shaped pipe 46 is fixedly connected to the shell 1 and the return ring 41, and the filter screen 40 is capable of filtering impurities.

A second sealing ring 44 is attached to the return ring 41. The second sealing ring 44 is in contact with the return pipe 42, and the second sealing ring 44 is capable of improving the sealing performance.

The return pipe 42 is fixedly connected to the applying shell 2, and the sealing plate 45 is fixedly connected to the interior of the applying shell 2. The sealing plate 45 and the return pipe 42 are both in contact with the applying sponge 33, the return pipe 42 is fixedly connected to the sealing plate 45, and the return pipe 42 is capable of sucking the liquid medicine from the applying sponge 33.

The power supply mechanism 5 comprises a power supply handle compartment 51, a conductive plate 52, a second spring 53, a conductive cover 54, a conductive elastic piece 55, a screw 56, a baffle 57 and a dry battery compartment 58. The power supply handle compartment 51 is fixedly connected to an outer side of the shell 1, the conductive plate 52 is slidably connected to the interior of the power supply handle compartment 51, and the second spring 53 is arranged on the conductive plate 52. The conductive cover 54 is hinged to the power supply handle compartment 51, the conductive elastic piece 55 is fixedly connected to the conductive cover 54, and the screw 56 is slidably connected to the interior of the conductive cover 54. The baffle 57 is fixedly connected to an outer side of the screw 56, the screw 56 is in threaded connection with the power supply handle compartment 51, and the baffle 57 is in contact with the power supply handle compartment 51. The dry battery compartment 58 is slidably connected to the interior of the power supply handle compartment 51, and the dry battery compartment 58 is in contact with the conductive plate 52 and the conductive elastic piece 55. One end of the second spring 53 is fixedly connected with the power supply handle compartment 51, the other end of the second spring 53 is fixedly connected to the conductive plate 52, and the baffle 57 is capable of preventing the screw 56 from being disengaged from the conductive cover 54.

Before use, the disinfected applying shell 2 is taken out, and then the driving ring 34 is pressed towards the interior of the infusion pipe 31. When the inclined surface of the driving ring 34 is separated from the inclined surface block 38, the compressed corrugated pipe 37 propels the connecting column 36 to reset through the elastic force. After the input pipe 13 is inserted into the infusion pipe 31, the driving ring 34 is released, the first spring 35 is reset to propel the driving ring 34 to slide through the elastic force, and the driving ring 34 propels the connecting column 36 to slide into the input pipe 13 through the inclined surface block 38. Subsequently, the threaded cover 43 on the outer side of the return pipe 42 is screwed to the outer side of the return ring 41, thereby completing the assembly of the medicine applying device of the present invention.

When in use, the applying shell 2 is placed into a patient's esophagus, and the camera 7 is initiated to search for a wound surface. After the applying shell 2 moves to the wound surface, the output pump 12 is initiated. At this point, the output pump 12 discharges the liquid medicine in the shell 1 into the interior of the infusion pipe 31 through the input pipe 13, and the liquid medicine in the infusion tube 31 is discharged into the interior of the applying sponge 33 through the through hole 32. After the liquid medicine is discharged, the return pump 49 is initiated to generate a suction force, and the suction force is transferred to the interior of the return ring 41 via the Y-shaped pipe 46. The suction force in the return ring 41 sucks the liquid medicine from the applying sponge 33 through the return pipe 42 such that the amount of the medicine is reduced, which effectively prevents the excessive liquid medicine from flowing into the stomach along the esophagus.

When the power of the medicine applying device becomes insufficient, the screw 56 in the power supply handle compartment 51 is removed, and the conductive cover 54 is turned downwards, which allow the dry battery compartment 58 to be taken out for replacement. After the old dry battery compartment 58 is removed, a new dry battery compartment 58 is placed into the power supply handle compartment 51. Subsequently, the screw 56 is screwed into the interior of the power supply handle compartment 51, thereby completing the replacement of the dry battery compartment 58.

The basic principles, main features and advantages of the present invention are described in the above. Those skilled in the art should understand that the present invention is not limited by the above embodiments. The above embodiments and instructions are merely used to describe the principle of the present invention. Various modifications and improvements may be made by those skilled in the art without departing from the spirit of the present invention, and all of which shall therefore fall into the scope of the present invention. The scope of the present invention is defined by the claims of the present invention.

1. A esophagus medicine applying device, comprising:
a shell (1),
an applying shell (2),
an assembling mechanism (3),
a return mechanism (4),
a power supply mechanism (5),
an arc cover (6), p1 a camera (7),
a transparent cover (8), and
an end cover (9), wherein the return mechanism (4) is arranged within the shell (1), wherein the assembling mechanism (3) is arranged on the applying shell (2), wherein the power supply mechanism (5) is arranged on the shell (1), wherein the arc cover (6) is connected to the applying shell (2) through threads, wherein the camera (7) is fixedly connected to the interior of the arc cover (6), wherein the transparent cover (8) is fixedly connected to the arc cover (6), wherein one end of the shell (1) is in threaded connection with the end cover (9), wherein a partition plate (10) is fixedly connected to the interior of the shell (1), wherein an input pipe (13) is fixedly connected to the interior of the shell (1), and wherein an output pump (12) is fixedly connected to the partition plate (10).

2. The esophagus medicine applying device of claim 1, wherein the assembling mechanism (3) further comprising:
an infusion pipe (31), a through hole (32), an applying sponge (33), a driving ring (34), a first spring (35), a connecting column (36), a corrugated pipe (37), an inclined surface block (38) and a first sealing ring (39), wherein the infusion pipe (31) is fixedly connected to the interior of the applying shell (2), wherein the through hole (32) is formed in the infusion pipe (31), wherein the applying sponge (33) is fixedly connected to the outer side of the infusion pipe (31), wherein the applying sponge (33) is in contact with the applying shell (2), wherein the driving ring (34) is slidably connected to the infusion pipe (31), wherein the first spring (35) is arranged on the driving ring (34), the connecting column (36) is slidably connected to the interior of the infusion pipe (31), wherein the inclined surface block (38) is fixedly connected to the connecting column (36), wherein the inclined surface block (38) is slidably connected to the driving ring (34), wherein the corrugated pipe (37) is arranged on an outer side of the connecting column (36), wherein the first sealing ring (39) is attached to an interior of the infusion pipe (31), wherein the first sealing ring (39) is in contact with the input pipe (13), wherein the input pipe (13) is slidably connected to the infusion pipe (31), and wherein the infusion pipe (31) is in contact with the inclined surface block (38).

3. The esophagus medicine applying device of claim 2, wherein one end of the first spring (35) is fixedly connected to the driving ring (34), and another end of the first spring (35) is fixedly connected to the infusion pipe (31).

4. The esophagus medicine applying device of claim 2, wherein one end of the corrugated pipe (37) is fixedly connected to the inclined surface block (38), and another end of the corrugated pipe (37) is fixedly connected to the infusion pipe (31).

5. The esophagus medicine applying device of claim 1, wherein the return mechanism (4) further comprising:
a return ring (41), a return pipe (42), a threaded cover (43), a second sealing ring (44), a sealing plate (45), a Y-shaped pipe (46), a threaded pipe (47), a protective cover (48), a return pump (49) and a filter screen (40), wherein the return ring (41) is fixedly connected to the shell (1), wherein the return pipe (42) is slidably connected to the return ring (41), wherein the threaded cover (43) is movably connected to an outer side of the return pipe (42), wherein the threaded cover (43) is in threaded connection with the return ring (41), wherein the Y-shaped pipe (46) is fixedly connected to the partition plate (10), wherein the threaded pipe (47) is connected to an outer side of the Y-shaped pipe (46) through threads, wherein the return pump (49) is fixedly connected to the threaded pipe (47), wherein the return pump (49) is fixedly connected to the Y-shaped pipe (46), wherein the protective cover (48) is fixedly connected to an outer side of the threaded pipe (47), wherein the filter screen (40) is fixedly connected to the protective cover (48), and wherein the Y-shaped pipe (46) is fixedly connected to the shell (1) and the return ring (41).

6. The esophagus medicine applying device of claim 5, wherein a second sealing ring (44) is attached to the return ring (41), wherein the second sealing ring (44) is in contact with the return pipe (42).

7. The esophagus medicine applying device of claim 2, wherein the return pipe (42) is fixedly connected to the applying shell (2), wherein the sealing plate (45) is fixedly connected to the interior of the applying shell (2), wherein the sealing plate (45) and the return pipe (42) are both in contact with the applying sponge (33), and wherein the return pipe (42) is fixedly connected to the sealing plate (45).

8. The esophagus medicine applying device of claim 1, wherein the power supply mechanism (5) further comprising:
A power supply handle compartment (51), a conductive plate (52), a second spring (53), a conductive cover (54), a conductive elastic piece (55), a screw (56), a baffle (57) and a dry battery compartment (58), wherein the power supply handle compartment (51) is fixedly connected to an outer side of the shell (1), wherein the conductive plate (52) is slidably connected to an interior of the power supply handle compartment (51), wherein the second spring (53) is arranged on the conductive plate (52), wherein the conductive cover (54) is hinged to the power supply handle compartment (51), wherein the conductive elastic piece (55) is fixedly connected to the conductive cover (54), wherein the screw (56) is slidably connected to the interior of the conductive cover (54), wherein the baffle (57) is fixedly connected to an outer side of the screw (56), wherein the screw (56) is in threaded connection with the power supply handle compartment (51), wherein the baffle (57) is in contact with the power supply handle compartment (51), wherein the dry battery compartment (58) is slidably connected to the interior of the power supply handle compartment (51), wherein the dry battery compartment (58) is in contact with the conductive plate (52) and the conductive elastic piece (55), wherein one end of the second spring (53) is fixedly connected with the power supply handle compartment (51), and wherein another end of the second spring (53) is fixedly connected to the conductive plate (52).
